(19) United States
(12) Patent Application Publication
(10) Pub. No.: US 2020/0085374 A1
(43) Pub. Date: Mar. 19, 2020

(54) HEART RATE MEASUREMENT METHOD AND DEVICE, AND WEARABLE APPARATUS

(71) Applicants: BOE TECHNOLOGY GROUP CO., LTD., Beijing (CN); BEIJING BOE MULTIMEDIA TECHNOLOGY CO., LTD., Beijing (CN)

(72) Inventors: Sen LIN, Beijing (CN); Quanzhong WANG, Beijing (CN)

(73) Assignees: BOE TECHNOLOGY GROUP CO., LTD., Beijing (CN); BEIJING BOE MULTIMEDIA TECHNOLOGY CO., LTD., Beijing (CN)

(21) Appl. No.: 15/747,679

(22) PCT Filed: Aug. 22, 2017
(86) PCT No.: PCT/CN2017/098472

§ 371 Date: Jan. 25, 2018

(30) Foreign Application Priority Data
Aug. 26, 2016 (CN) 201610744267.0

(51) Int. Cl. A61B 5/00; A61B 5/0205; A61B 5/024

(52) U.S. Cl. CPC A61B 5/6844; A61B 5/02055; A61B 5/02416; A61B 5/7207; A61B 5/7405; A61B 5/681; A61B 5/7455; A61B 5/742; A61B 5/02438

(57) ABSTRACT

A heart rate measurement method and device is disclosed. A heart rate detecting sensor detects one biometric signal (e.g. PPG) of a user at a first time interval. A fitness detecting sensor detects a distance signal reflecting and corresponding to a degree of fitness between the heart rate measurement device and skin of the user. A processor obtains a heart rate value based on the one biometric signal, determines whether the heart rate value is desirable based on the distance signal. A heart rate value is kept if determined to be desirable, or discarded if otherwise. Physiological information of the user can be collected to determine whether a body of the user is in a state of movement, and if so, an action instruction can be sent to the heart rate measurement device to adjust the degree of fitness between the device and skin of the user.

### CROSS-REFERENCE TO RELATED APPLICATION

The present application claims priority to Chinese Patent Application No. 201610744267.0 filed on Aug. 26, 2016, the disclosure of which is hereby incorporated by reference in its entirety.

### TECHNICAL FIELD

The present disclosure is related generally to electronics technologies, and more specifically to a heart rate measurement method, a heart rate measurement device, and a wearable apparatus.

### BACKGROUND

With the improvement of quality of life, people are increasingly concerned about the health statuses of themselves, and correspondingly a variety of health care monitoring products are emerging on the market. As one of crucial physiological parameters, the heart rate of an individual has received a lot of attention, and has represented one of the most frequent health care monitoring targets.

On the other hand, smart electronics devices for terminal use, such as smart bracelets, smart watches, smart pendants, as well as other smart items convenient for wearing and carrying on, are also getting more and more favor on the market.

Currently on the market, there has been a trend to integrate the health care monitoring products and the smart electronics devices together, in order to realize an intelligent monitoring of the health status of a user, and to obtain real-time health data as well.

Heart rate monitoring has been a crucial part for monitoring of the health status of a user, and currently there have been a variety of different methods for measuring and monitoring heart rates of an individual. These methods include electrocardiography (ECG), whereby the bio-potential generated by electrical signals that control the expansion and contraction of heart chambers are measured, photoplethysmography (PPG), wherein a light-based technology is utilized to sense the rate of blood flow as controlled by the heart's pumping action, pulse oximetry, whereby a person's blood oxygen saturation is monitored by a pulse oximeter to thereby obtain a heart rate of the person, and an arterial pressure approach, whereby the heart rate is determined by measuring an arterial pressure of an individual.

The current photoplethysmography (PPG)-based heart rate measurement and detection device, because of its reliability in detection, small sizes of the circuit area and of the sensor electrode, no need for conductive media, and flexibility in deployment, has received a lot of attention, and has been widely employed in smart wristbands, smart watches, and other smart wearable apparatuses on the market.

The working principle of PPG detection of a heart rate detection device is as follows: a light-emitting sub-device first sheds a light beam of a certain wavelength to the blood vessels under the skin, then optical signals carrying information of the blood vessels and blood flow conditions are reflected or transmitted to a light-receiving sub-device for subsequent detection.

If a relatively large amount of blood is flowing in a blood vessel, the absorption of light by the blood and the blood vessel is also large, which in turn causes the light reflected or refracted by the blood vessel that reaches the light receiving sub-device has a relatively weak intensity. On the other hand, if a relatively small amount of blood is flowing in the blood vessel, the reflected or refracted light has a relatively strong intensity. By means of the corresponding relationship between heart rates and the intensity of the light received by the light receiving sub-device, the heart rate data can be obtained and determined.

There are a variety of movements of a human body, such as walking, jogging, and running, etc., and at a different movement state, the human body and the intelligent monitoring device have a different degree of fitness.

The PPG-based heart rate measurement and detection device and method has a higher requirement for fitness between the intelligent monitoring device and the human skin. When the degree of fitness is relatively low, it is susceptible to interference from lights from the outer environment, which negatively affects the accuracy of the monitoring data.

Currently, however, existing smart wearable apparatuses having a heart rate measurement device cannot judge the accuracy of the data detected thereby because it cannot recognize the degree of fitness between the smart monitoring device and the skin of a user, and is thus incapable of providing accurate and real-time monitoring results for the user.

### SUMMARY

In order to address the issues associated with the heart rate measurement and detection method that have been employed in current intelligent monitoring devices, the present disclosure provides a heart rate measurement method, a heart rate measurement device, and a wearable apparatus, which can be employed for accurate and real-time monitoring of the heart rates for a user.

In a first aspect, a method for monitoring heart rates of a user is disclosed herein. The user can be wearing a heart rate measurement device that is configured to detect biometric signals of the user, each at a first time interval. Each time when a biometric signal is to be detected, the method comprises the following steps:

detecting one biometric signal of the user and a distance signal reflecting and corresponding to a degree of fitness between the heart rate measurement device and skin of the user;

obtaining a heart rate value based on the one biometric signal, and determining whether the heart rate value is desirable based on the distance signal; and

keeping the heart rate value if the heart rate value is determined to be desirable, or discarding the heart rate value if otherwise.

Herein the biometric signals are at least one of photoplethysmography (PPG) signals, electrocardiography (ECG) signals, blood oxygen saturation signals, or arterial pressure signals. In some preferred embodiment, the biometric signals are PPG signals.

According to some embodiments of the method, in the step of keeping the heart rate value if the heart rate value is determined to be desirable, or discarding the heart rate value if otherwise, the discarding the heart rate value includes the following sub-steps:

starting timing; and

sending an action instruction to the heart rate measurement device to adjust the degree of fitness between the heart rate measurement device and the skin of the user if no heart rate value is determined as desirable within a time period, or stopping timing and resetting if otherwise.

In the method as described above, the action instruction can include a prompt instruction configured to prompt the user for manually adjusting the heart rate measurement device to be fit with the skin of the user. As such, the sub-step of sending an action instruction to the heart rate measurement device to adjust the degree of fitness between the heart rate measurement device and the skin of the user can include:

sending the prompt instruction to the heart rate measurement device.

In the method as described above, the action instruction can include an adjusting instruction configured to instruct the heart rate measurement device for automatically adjusting to be fit with the skin of the user. As such, the sub-step of sending an instruction to the heart rate measurement device to adjust the degree of fitness between the heart rate measurement device and the skin of the user can include:

sending the adjusting instruction to the heart rate measurement device.

According to some embodiments of the method prior to the step of detecting one biometric signal of the user and a distance signal reflecting and corresponding to a degree of fitness between the heart rate measurement device and skin of the user, the method can further include the following steps:

collecting physiological information of the user;

determining whether a body of the user is in a state of movement based on the physiological information; and

sending an action instruction to the heart rate measurement device to adjust the degree of fitness between the heart rate measurement device and the skin of the user if the body of the user is determined to be in the state of movement.

In the method as described above, the action instruction can include a prompt instruction configured to prompt the user for manually adjusting the heart rate measurement device to be fit with the skin of the user. As such, the sub-step of sending an action instruction to the heart rate measurement device to manually or automatically adjust the degree of fitness between the heart rate measurement device and the skin of the user can include:

sending the prompt instruction to the heart rate measurement device.

In the method as described above, the action instruction can include an adjusting instruction configured to instruct the heart rate measurement device for automatically adjusting to be fit with the skin of the user. As such, the sub-step sending an instruction to the heart rate measurement device to manually or automatically adjust the degree of fitness between the heart rate measurement device and the skin of the user can include:

sending the adjusting instruction to the heart rate measurement device.

In the method as described above, the distance signal can include at least one of a temperature signal, an optical pulse signal, or a second time interval signal.

According to some embodiments of the method where the distance signal includes a temperature signal. As such, the step of detecting one biometric signal of the user and a distance signal reflecting and corresponding to a degree of fitness between the heart rate measurement device and skin of the user can include:

detecting an instant temperature of the user by a temperature sensor;

In addition, the step of obtaining a heart rate value based on the one biometric signal and determining whether the heart rate value is desirable based on the distance signal can include the following sub-steps:

obtaining a change of temperature by comparing the instant temperature with a reference temperature; and

determining that the heart rate value is not desirable if the change of temperature is greater than a preset threshold, or determining that the heart rate value is desirable if otherwise.

According to some other embodiments of the method where the distance signal includes an optical pulse signal, the step of detecting one biometric signal of the user and a distance signal reflecting and corresponding to a degree of fitness between the heart rate measurement device and skin of the user can include:

detecting an instant optical pulse signal reflected from the skin of the user by a proximity sensor.

In addition, the step of obtaining a heart rate value based on the one biometric signal and determining whether the heart rate value is desirable based on the distance signal can include the following sub-steps:

obtaining a change of optical pulse signal by comparing the instant optical pulse signal with a reference optical pulse signal; and

determining that the heart rate value is not desirable if the change of optical pulse signal is greater than a preset threshold, or determining that the heart rate value is desirable if otherwise.

According to yet some other embodiments of the method where the distance signal includes a second time interval signal, the step of detecting one biometric signal of the user and a distance signal reflecting and corresponding to a degree of fitness between the heart rate measurement device and skin of the user can include:

detecting an instant second time interval signal of an infrared light reflected from the skin of the user by a distance sensor;

In addition, the step of obtaining a heart rate value based on the one biometric signal and determining whether the heart rate value is desirable based on the distance signal can include the following sub-steps:

comparing the instant second time interval signal with a preset threshold; and

determining that the heart rate value is not desirable if the instant second time interval signal is greater than the preset threshold, or determining that the heart rate value is desirable if otherwise.

In a second aspect, the disclosure further provides a heart rate measurement device.

The heart rate measurement device includes a heart rate detecting sensor, a fitness detecting sensor, and a processor. The heart rate detecting sensor is coupled to the processor, and is configured to detect, and to transmit to the processor, one biometric signal of a user at a first time interval; the fitness detecting sensor is coupled to the processor, and is configured to detect, and to transmit to the processor, a distance signal corresponding to the one biometric signal, wherein the distance signal reflects and corresponds to a degree of fitness between the heart rate measurement device and the skin of the user; and the processor is configured to obtain a heart rate value based on the one biometric signal, and to determine whether the heart rate value is desirable based on the distance signal.

The heart rate measurement device can further include a memory, which is coupled to the processor and is configured to store the heart rate value if the heart rate value is determined by the processor to be desirable.

According to some embodiments, the heart rate measurement device further includes an action portion coupled to the processor, and as such, the processor is further configured to start timing if determining that the heart rate value is not desirable, and to send an action instruction to the action portion if no heart rate value is determined as desirable within a time period, or to stop timing and reset if otherwise; and the action portion is configured, upon receiving the action instruction from the processor, to act on the heart rate measurement device to adjust the degree of fitness between the heart rate measurement device and the skin of the user.

In the heart rate measurement device as described above, the action portion can include a prompting portion, and accordingly the action instruction comprises a prompt instruction. The prompting portion is configured, upon receiving the prompt instruction from the processor, to prompt the user for manually adjusting the heart rate measurement device to be fit with the skin of the user.

In the heart rate measurement device as described above, the action portion can include an adjusting portion, and accordingly the action instruction comprises an adjusting instruction. The adjusting portion is configured, upon receiving the adjusting instruction from the processor, to automatically adjust the heart rate measurement device to be fit with the skin of the user.

According to some embodiments of the disclosure, the heart rate measurement device further includes a physiological information collecting portion and an action portion, each coupled to the processor.

The physiological information collecting portion is configured to collect physiological information of the user, and to send the physiological information to the processor.

Accordingly, the processor is further configured to determine whether a body of the user is in a state of movement based on the physiological information, and if so, to start timing and to send an action instruction to the action portion, or if otherwise, to stop timing and reset.

Furthermore, the action portion is configured, upon receiving the action instruction from the processor, to act on the heart rate measurement device to adjust the degree of fitness between the heart rate measurement device and the skin of the user.

In the heart rate measurement device as described above, the action portion can comprise a prompting portion, and accordingly the action instruction comprises a prompt instruction. As such, the prompting portion is configured, upon receiving the prompt instruction from the processor, to prompt the user for manually adjusting the heart rate measurement device to be fit with the skin of the user.

In the heart rate measurement device as described above, the action portion can include an adjusting portion, and accordingly the action instruction comprises an adjusting instruction. As such, the adjusting portion is configured, upon receiving the adjusting instruction from the processor, to automatically adjust the heart rate measurement device to be fit with the skin of the user.

In the heart rate measurement device, the prompting portion can include at least one of a display sub-portion, a vibration sub-portion, an audio sub-portion, or a light-emitting sub-portion.

In some embodiments of the heart rate measurement device, the prompting portion includes a display sub-portion, configured to display the heart rate value.

According to some other embodiments of the disclosure, the adjusting portion includes a wearing sub-portion and a fastening sub-portion. The wearing sub-portion is configured to attach the heart rate measurement device on a part of the human body of the user; and the fastening sub-portion is disposed on the wearing sub-portion and is configured to fasten or loosen the wearing sub-portion to thereby adjust a degree of fitness between the heart rate measurement device and the skin of the user based on the adjusting instruction from the processor.

In the heart rate measurement device as described above, the fitness detecting sensor can include at least one of a temperature sensor, a proximity sensor, or a distance sensor.

In the embodiments of the heart rate measurement device where the fitness detecting sensor includes a temperature sensor, the temperature sensor is configured to detect an instant temperature of the user, and the processor is further configured to obtain a change of temperature by comparing the instant temperature with a reference temperature, and to determine that the heart rate value is not desirable if the change of temperature is greater than a preset threshold, or that the heart rate value is desirable if otherwise.

In the embodiments of the heart rate measurement device where the fitness detecting sensor includes a proximity sensor, the proximity sensor is configured to detect an instant optical pulse signal reflected from the skin of the user, and the processor is further configured to obtain a change of optical pulse signal by comparing the instant optical pulse signal with a reference optical pulse signal, and to determine that the heart rate value is not desirable if the change of optical pulse signal is greater than a preset threshold, or that the heart rate value is desirable if otherwise.

In the embodiments of the heart rate measurement device where the fitness detecting sensor includes a distance sensor, the distance sensor is configured to detect an instant second time interval signal of an infrared light reflected from the skin of the user, and the processor is further configured to compare the instant second time interval signal with a preset threshold, and to determine that the heart rate value is not desirable if the instant second time interval signal is greater than the preset threshold, or that the heart rate value is desirable if otherwise.

In a third aspect, a wearable apparatus is disclosed, which includes a heart rate measurement device according to any of the embodiments as described above. The heart rate measurement device is disposed on a side of a user in contact with the skin of the user.

In the heart rate measurement device and method disclosed herein, a fitness detecting sensor is arranged in the heart rate measurement device, and is configured to detect a distance between the heart rate measurement device, which reflects a degree of fitness between the heart rate measurement device and skin of a user. The distance is then transmitted from the fitness detecting sensor to the processor,

The processor is configured to determine if the heart rate measurement device is fit with skin of a user, and based on the above determination result, to determine if the heart rate value obtained based on a PPG signal detected by the heart rate detecting sensor is desirable. The heart rate value can be stored in the memory if so, and can be removed if otherwise.

As such, inaccurate heart rate values can be removed, and the heart rate values that have been stored in the memory are each accurate heart rate value, thereby improving the heart rate detecting result.

### BRIEF DESCRIPTION OF THE DRAWINGS

To more clearly illustrate some of the embodiments, the following is a brief description of the drawings. The drawings in the following descriptions are only illustrative of some embodiment. For those of ordinary skill in the art, other drawings of other embodiments can become apparent based on these drawings.

FIG. 1 illustrates a schematic diagram of a heart rate measurement device according to a first embodiment of the present disclosure;

FIG. 2 illustrates a schematic diagram of a heart rate measurement device according to a second embodiment of the present disclosure;

FIG. 3 illustrates a schematic diagram of a heart rate measurement device according to a third embodiment of the present disclosure;

FIG. 4 illustrates a schematic diagram of a heart rate measurement device according to a fourth embodiment of the present disclosure;

FIG. 5 illustrates a schematic diagram of a heart rate measurement device according to a fifth embodiment of the present disclosure;

FIG. 6A illustrates a schematic diagram of a heart rate measurement device according to a sixth embodiment of the present disclosure;

FIG. 6B illustrates a schematic diagram of an adjusting portion in the heart rate measurement device when it is at a fastening state;

FIG. 6C illustrates a schematic diagram of an adjusting portion in the heart rate measurement device when it is at a loosening state;

FIG. 6D illustrates a schematic diagram of an adjusting portion in the heart rate measurement device according to some embodiments of the present disclosure;

FIG. 7 illustrates a schematic diagram of connections among various components of a heart rate measurement device according to a seventh embodiment of the present disclosure;

FIG. 8 shows a flow chart of a method for measuring a heart rate of a user by means of a heart rate measurement device according to some embodiments of the present disclosure;

FIG. 9 shows a flow chart of a method for measuring a heart rate of a user by means of a heart rate measurement device according to some other embodiments of the present disclosure.

### DETAILED DESCRIPTION

In the following, with reference to the drawings of various embodiments disclosed herein, the technical solutions of the embodiments of the disclosure will be described in a clear and fully understandable way.

It is obvious that the described embodiments are merely a portion but not all of the embodiments of the disclosure. Based on the described embodiments of the disclosure, those ordinarily skilled in the art can obtain other embodiment(s), which come(s) within the scope sought for protection by the disclosure.

In a first aspect, the present disclosure provides a heart rate measurement device. The heart rate measurement device can be an independent device worn by a user and specifically configured for measuring heart rates of a user, but can also be an functional module in a wearable apparatus worn by an individual that has a functionality of measuring heart rates for the user.

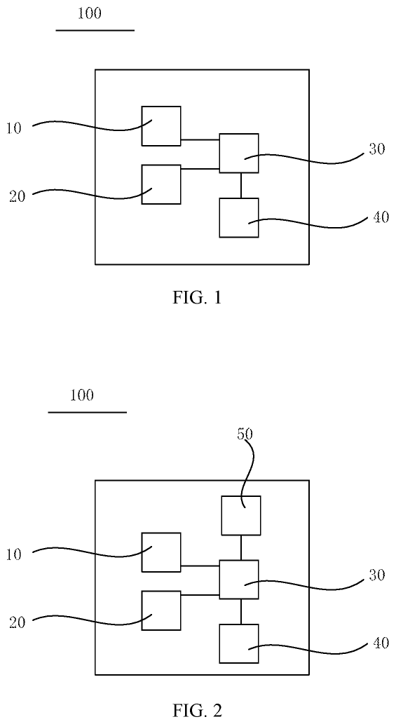

FIG. 1 illustrates a schematic diagram of a heart rate measurement device according to a first embodiment of the present disclosure. As shown in FIG. 1, the heart rate measurement device 100 comprises a heart rate detecting sensor 10, a fitness detecting sensor 20, a processor 30, and a memory 40.

The heart rate detecting sensor 10 is configured to detect, and to transmit to the processor 10, a photoplethysmography (PPG) signal in a body of a user.

The fitness detecting sensor 20 is configured to detect, and to transmit to the processor 10, a distance signal comprising a value of distance between the heart rate measurement device 100 and a human skin of the user. Herein the value of distance between the heart rate measurement device 100 and the human skin of the user is configured to reflect, or correspond to, a degree of fitness between the heart rate measurement device 100 and the human skin of the user.

The processor 30 is configured to obtain a heart rate value based on the PPG signal transmitted from the heart rate detecting sensor 10, to determine whether the heart rate value is desirable based on the distance value transmitted from the fitness detecting sensor 20, and to store the heart rate value in the memory 40 if so, or to remove the heart rate value if otherwise.

The following are noted.

First, there is no limitation to the specific types or structures of the heart rate measurement device 100, as long as the heart rate measurement device 100 touches the human skin after wearing.

Second, in this specific embodiment as described above, the heart rate detecting sensor 10 utilizes a PPG—based heart rate determining method for the determination of a heart rate of a user. The heart rate detecting sensor 10 can also utilize other methods, including an ECG-based, a pulse oximetry-based, or an arterial pressure-based heart rate determining method for the determination of a user's heart rate.

Third, there is no limitation to the specific types or structures of the fitness detecting sensor 20, as long as the fitness detecting sensor 20 can transmit to the processor 30 a signal corresponding to a degree of fitness between the heart rate measurement device 100 and the human skin.

Fourth, it is further noted that in addition to the PPG-based method that is utilized by the heart rate detecting sensor 10 in the heart rate measurement device 100 for the determination of a user's heart rate, other methods, including an ECG-based heart rate determining method, a pulse oximetry-based heart rate determining method, or an arterial pressure-based heart rate determining method, can also be utilized by the heart rate detecting sensor 10 in the heart rate measurement device 100 for determining a heart rate of the user. For convenience and simplicity, only the PPG-based method is described in detail as an illustrating example in the disclosure.

Herein the processor 30 can determine whether or not the heart rate measuring device 100 is fit with the human skin of the user based on the distance value transmitted from the fitness detecting sensor 20.

If the heart rate measuring device 100 is determined not to be fit with the human skin based on the distance signal, the heart rate values that are obtained after calculation over the PPG signal transmitted from the heart rate detecting sensor 10 are determined as not desirable.

If, on the other hand, the heart rate measuring device 100 is determined to be fit with the human skin, the heart rate values that are obtained after calculation over the PPG signal transmitted from the heart rate detecting sensor 10 are determined as desirable.

The processor 30 can be arranged on a printed circuit board (PCB), and can be a master chip. Herein the PCB can be arranged on the heart rate measurement device 100. Other embodiments are also possible.

The memory 40 can be integrated into the processor 30, but can also function as a component of the heart rate measurement device 100. Alternatively, the memory 40 can be realized by externally inserting a memory card into a slot that has been arranged. Other embodiments are possible, and there are no limitations herein.

In the above embodiment of the heart rate measurement device 100, the heart rate detecting sensor 10, the fitness detecting sensor 20, the processor 30, and the memory 40 all need a power supply to work functionally. As such, the heart rate measuring device 100 can further include a power supply (not shown), which can be arranged on a PCB, or may be placed elsewhere in other places according to the structure of the heart rate measuring device 100, as long as the power supply can supply power to the various components in the heart rate measuring device 100.

Specifically, if the power supply is a battery, it may be disposed inside the heart rate measuring device 100, or inside another electronics apparatus (such as a wearable apparatus) that also comprises the heart rate measurement device 100. If the power supply is a solar cell, it may be disposed on an outer surface of the heart rate measuring device 100 so as to facilitate providing the power supply to the heart rate measurement device 100 at any time. It is also possible to provide a charging interface on the heart rate measuring device 100 through which the battery can be charged.

The embodiment as described above does not impose limitations to the specific configuration of the heart rate detecting sensor 10, the fitness detecting sensor 20, the processor 30, and the memory 40, which can be configured based on the structure of the heart rate measurement device 100.

In the above embodiment of the heart rate measurement device 100, a fitness detecting sensor 20 is arranged in the heart rate measurement device 100, and is configured to detect a distance signal that reflects and corresponds to a degree of fitness between the heart rate measurement device 100 and skin of a user. The distance signal is then transmitted to the processor 30.

By determining whether or not the heart rate measuring device 100 is fit with the human skin of the user, the processor 30 can determine whether or not heart rate values obtained by calculating the photoplethysmography (PPG) signal of the blood detected by the heart rate detecting sensor 10 is desirable.

If the heart rate measuring device 100 is not fit with the human skin of the user, the heart rate values that are obtained after calculation are determined as not reliable or desirable, and are then removed. If the heart rate measuring device 100 is fit with the human skin of the user, the heart rate values that are obtained after calculation are determined as reliable and desirable, and are then stored in the memory 40. .

As such, the heart rate values that are determined to be inaccurate can be removed, and the heart rate values stored in the memory 40 can only be accurate heart rate values, thereby improving the accuracy of the heart rate detection results.

FIG. 2 illustrates a schematic diagram of connections among various components of a heart rate measurement device according to a second embodiment of the present disclosure.

As shown in FIG. 2, the heart rate measurement device 100 further comprises a prompting portion 50, configured to prompt to the user that the heart rate measurement device 100 needs to be adjusted to be fit with the skin of the user.

The processor 30 is further configured to start timing if determining that the heart rate value is not desirable, and to send a prompt instruction to the prompting portion 50 if determining that none of the heart rate values collected by the heart rate detecting sensor 10 within a time period is desirable, or to stop timing and reset if determining that there is at least one desirable heart rate value within the time period. Herein, resetting refers to setting the processor to its original status (i.e. before determining whether there is at least one desirable heart rate value within a time period).

The prompting portion 50 is configured to send a prompt to the user based on the prompt instruction sent by the processor 30, which is purported to prompt the user that the heart rate measurement device 100 needs to be adjusted so as to be fit with the human skin.

Herein, the prompting portion 50 can comprise at least one of a display sub-portion, a vibration sub-portion, an audio sub-portion, or a light-emitting sub-portion, so as to meet the different requirements under a variety of different situations.

For example, if a display sub-portion is utilized in the prompting portion 50, the prompt can be displayed on a screen, and optionally, the heart rate values can be configured to be displayed on the screen as well.

Similarly, if the prompting portion 50 comprises a vibration sub-portion, an audio sub-portion, or a light-emitting sub-portion, the prompt can be a vibration cue, a sound cue, or an optical cue, that is presented to the user.

Upon receiving the prompt from the prompting portion 50, the user can manually adjust the heart rate measurement device 100 to be fit with the skin.

Herein by configuring a prompting portion 50 in the heart rate measurement device 100, if none of the heart rate values is determined as desirable within a time period, the prompting portion 50 sends a prompt to the user based on the prompt instruction sent from the processor 30, so that the user can adjust the heart rate measurement device 100 to be fit with the skin to thereby obtain the heart rate values accurately, realizing a real-time monitoring of the heart rate of the user.

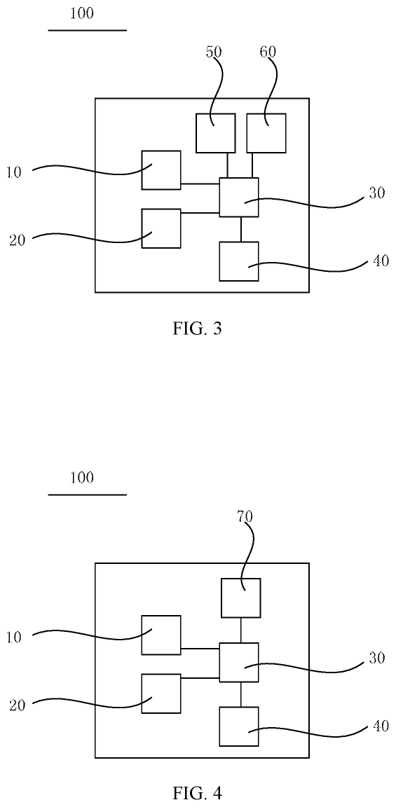

FIG. 3 illustrates a schematic diagram of a heart rate measurement device according to a third embodiment of the present disclosure. As shown in FIG. 3, the heart rate measurement device 100 further comprises a physiological information collecting portion 60.

The physiological information collecting portion 60 is configured to collect physiological information of the user in a real-time manner, and to send the physiological information to the processor 30. Correspondingly, the processor 30 is further configured to determine whether a body of the user is in a state of movement based on the physiological information collected from the physiological information collecting portion 60, and to send a prompt instruction to the prompting portion 50 if so.

Herein the physiological information that is collected by the physiological information collecting portion 60 can comprise a variety of information, such as pulse rate, body temperature, and heart beats, etc. The state of movement is referred to as a state of the user when the user is moving, and if the user is not moving, the user is not in the state of movement.

In an illustrating example where physiological information comprises body temperature, the physiological information collecting portion 60 comprises a thermometer. Because an individual typically has a different body temperature under a different state of movement, thus the processor 30 can perform an analysis over the body temperatures collected and sent by the physiological information collecting portion 60 to thereby determine the state of movement at the instant time.

Herein, by configuring a physiological information collecting portion 60 in the heart rate measurement device 100, the heart rate measurement device 100 can intelligently recognize a state of movement of the user, and can determine whether to send a prompt instruction to the prompting portion 50 based on the state of movement of the user so as to prompt the user to adjust the heart rate measurement device 100 to be fit with the skin to thereby obtain the heart rate values accurately.

FIG. 4 illustrates a schematic diagram of a heart rate measurement device according to a fourth embodiment of the present disclosure. As shown in FIG. 4, the heart rate measurement device 100 further comprises an adjusting portion 70, which is configured to adjust the heart rate measurement device 100 to be fit with the skin of the user based on an adjusting instruction from the processor 30.

The processor 30 is further configured to start timing if determining that the heart rate value is not desirable, and is configured to send the adjusting instruction to the adjusting portion 70 to decrease a distance between the heart rate measurement device 100 and the skin of the user if determining that none of the heart rate values is desirable within a time period, or to stop timing and reset if determining that there is at least one desirable heart rate value within the time period.

Herein there are no limitations to the specific structure of the adjusting portion 70, as long as the adjusting portion 70 can automatically adjust the heart rate measurement device 100 to be fit with the skin of the user.

Herein, by configuring an adjusting portion 70 in the heart rate measurement device 100, the adjusting portion 70 can automatically adjust the heart rate measurement device 100 to be fit with the skin of the user based on the adjusting instruction from the processor 30 so as to obtain accurate heart rate values to thereby realize a real-time monitoring of the heart rates of the user.

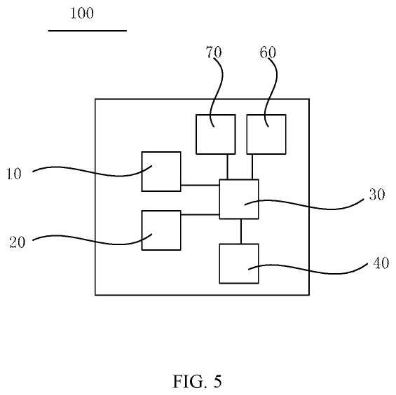

On top of the fourth embodiment of the heart rate measurement device 100 as shown in FIG. 4, the heart rate measurement device 100 further comprises a physiological information collecting portion 60, as illustrated in the fifth embodiment of the heart rate measurement device 100 shown in FIG. 5.

The physiological information collecting portion 60 is configured to collect physiological information of the user in a real-time manner, and to send the physiological information to the processor 30. The processor 30 is further configured to determine whether a human body of the user is in a state of movement based on the physiological information collected by the physiological information collecting portion 60, and if so, to send an adjusting instruction to the adjusting portion 70 so as to decrease a distance between the heart rate measurement device 100 and the skin of the user.

Herein, by configuring a physiological information collecting portion 60 in the heart rate measurement device 100, the heart rate measurement device 100 can intelligently recognize a state of movement of the user, and can determine whether to send an adjusting instruction to the adjusting portion 70 based on the state of movement of the user so as to automatically adjust a distance, and to thereby ensure a fitness, between the heart rate measurement device 100 and the skin of the user, thereby obtaining the heart rate values accurately.

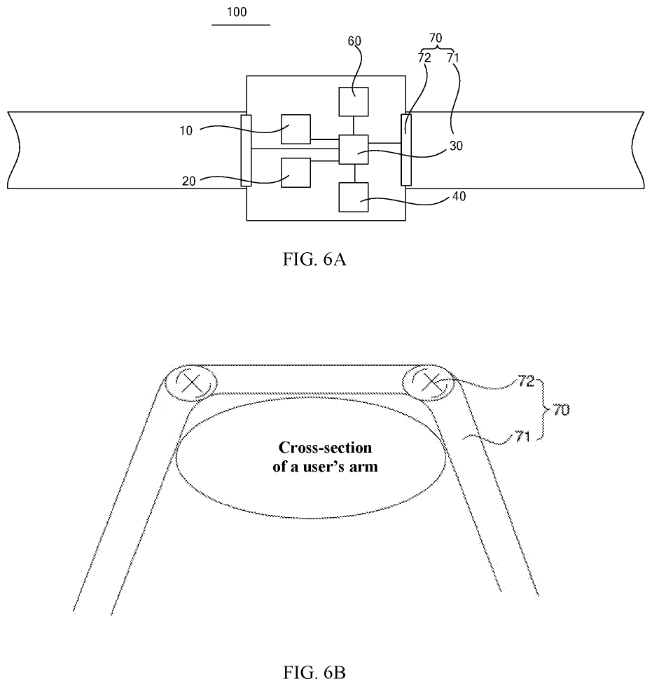

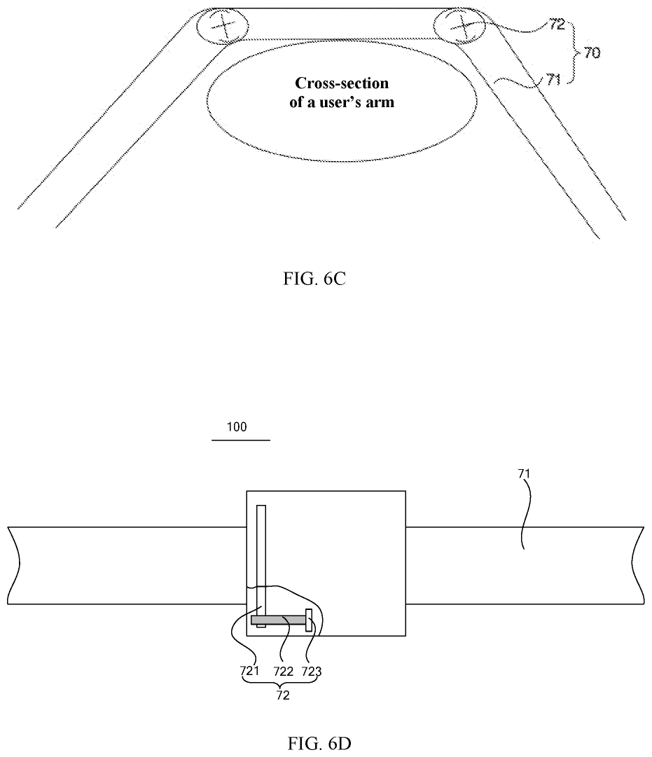

FIGS. 6A-6D illustrates several embodiments of the adjusting portion 70 of the heart rate measurement device 100. As shown in these figures, the adjusting portion 70 comprises a wearing sub-portion 71, and a fastening sub-portion 72 disposed on the wearing sub-portion 71.

The wearing sub-portion 71 is configured to attach the heart rate measurement device 100 on a part of the human body of the user. For example, the wearing sub-portion 71 can be a band, that is worn on a wrist, an ankle, an arm, a leg etc. of the user. The wearing sub-portion 71 can also be a patch with the heart rate measurement device 100, that is attached on a piece of skin on certain part of the body, such as a chest of the user.

The fastening sub-portion 72 is configured to fasten or loosen the wearing sub-portion 71 to thereby adjust a degree of fitness between the heart rate measurement device 100 and the skin of the user based on the adjusting instruction from the processor 30.

Herein there are no limitations to the specific structure and/or the number of the fastening sub-portion 72. There can be one or multiple fastening sub-portions 72 in different embodiment of the heart rate measurement device 100. For example, the embodiments of the heart rate measurement device 100 as illustrated in FIGS. 6A-6C have two fastening sub-portions 72.

As illustrated in FIG. 6D, in some embodiments of the present disclosure, the fastening sub-portion 72 can comprise a worm 721, a worm wheel 722, and a motor 723. The worm 721 is connected to the wearing sub-portion 71, and the worm wheel 722 is connected to the worm 721. The motor 723 is configured to rotate the worm wheel 722.

Specifically, the processor 30 sends the adjusting instruction to the fastening sub-portion 72 to thereby start the motor 723, which in turn drives the worm wheel 722 to rotate. Rotation of the worm wheel 722 in turn drives the worm 721 to rotate so as to realize a fastening or loosening of the wearing sub-portion 71. The fastening state and the loosening state of the wearing sub-portion 71 are respectively shown in FIG. 6B and FIG. 6C.

Herein, by means of the fastening sub-portion 72, which is further configured to fasten or loosen the wearing sub-portion 71 based on the adjusting instruction from the processor 30, thereby realizing an intelligent adjustment of the degree of fitness between the heart rate measurement device 100 and the skin of the user.

In any of the embodiments as described above, the heart rate measurement device 100 can be a smart wristband, a smart footband, a smart neckband, or a smart wearable apparatus.

According to some embodiments, the fitness detecting sensor 20 comprises a temperature sensor, configured to detect a temperature which reflects and corresponds to a distance between the heart rate measurement device 100 and the skin of the user. The temperature sensor is disposed on a side of the heart rate measurement device 100 that is in contact with the skin of the user.

The temperature sensor can be a contact-type temperature detecting sub-device, which can be, for example, a thermistor. The thermistor utilizes the following principle: when a surface of the thermistor is in contact with a spot of a surface of an object with different temperatures, a resistance of the thermistor changes rapidly. After reading a real-time resistance of the thermistor through a circuit, the real-time resistance of the thermistor is then converted to thereby obtain an instant temperature of the spot of the surface of the object contacted by the thermistor.

The instant temperature is then transmitted to the processor 30, and the processor 30 obtains a temperature change by comparing the instant temperature with a reference temperature, wherein the reference temperature is a temperature of the body of the user when the heart rate measuring device 100 is closely fit with the human skin.

Because the body temperature typically does not have a great change even during an intense activity, thus if the temperature change obtained by the processor 30 is greater than a preset threshold, the processor 30 can determine that the heart rate measuring device 100 is not fit with the skin of the human body at the instant time.

Herein, by configuring a temperature sensor as the fitness detecting sensor 20 in the heart rate measurement device 100, on the one hand, the fitness detecting sensor 20 can be used to detect a distance between the heart rate measurement device 100 and the skin of the user, and on the other hand, the fitness detecting sensor 20 can be used to determine a health state of the user using the body temperature as one parameter for health of the user.

It is noted that the fitness detecting sensor 20 in the heart rate measurement device 100 can be other types of sensor, such as a proximity sensor or a distance sensor.

Specifically, a proximity sensor determines a distance between the proximity sensor and the skin of a user by transmitting an optical pulse of extremely short wavelength and measuring the intensity of the optical pulse that is reflected back. Thus after being reflected by the skin of the user, an instant optical pulse signal is received by a receiver in the proximity sensor, and the proximity sensor further sends the instant optical pulse signal to the processor 30.

The processor 30 compares the instant optical pulse signal with a reference optical pulse signal to thereby obtain a change of the optical pulse signal. The reference optical pulse signal is an optical pulse signal received by the receiver if the heart rate measurement device 100 is closely fit with the skin of the user.

Because of a relatively short wavelength, the optical pulse signal is unable to transmit to a relatively long distance. Thus if the heart rate measurement device 100 is not fit with, or is relatively far away from, the skin of a user, the receiver in the fitness detecting sensor 20 can only receive a relatively weak, or no, optical pulse signal.

As such, if the change of the optical pulse signal is greater than a preset threshold, the processor 30 can determine that the heart rate measurement device 100 and the skin of the user have a relatively low degree of fitness.

Specifically, a distance sensor transmits an infrared light and measures a distance between the distance sensor and the skin of a user by measuring a time interval for the infrared light to travel after emission until reflection back from an object. By measuring the time interval, a distance between the distance sensor and the skin of a user can be calculated.

After reflection by the skin of the user, the infrared light can be received by a receiver in the distance sensor. Then the distance sensor calculates a time interval between an emission of the infrared light and reception of the infrared light, and further sends the time interval to the processor 30.

The more distant the infrared light is from the skin of a user, the longer time interval for the infrared light to be reflected back after emission. As such, the processor 30 compares an instant time interval with a preset threshold, and if the instant time interval is greater than a preset threshold, the processor 30 can determine that there is a relatively low degree of fitness between the heart rate measurement device 100 and the skin of the user.

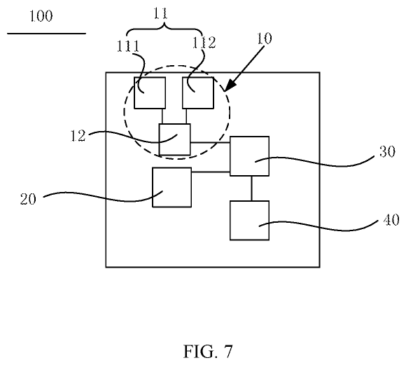

FIG. 7 illustrates a schematic diagram of a heart rate measurement device according to a seventh embodiment of the present disclosure. As shown in FIG. 7, the heart rate detecting sensor 10 of the heart rate measurement device 100 comprises a light-emitting circuit 11 and a light-receiving circuit 12.

The light-emitting circuit 11 comprises a first light-emitting sub-circuit 111 and a second light-emitting sub-circuit 112, wherein the first light-emitting sub-circuit 111 is configured to emit a green light, and the second light-emitting sub-circuit 112 is configured to emit a red light.

The light-emitting circuit 11 and the light-receiving circuit 12 can be disposed on a same side, or on opposing sides, of the heart rate measurement device 100. If the light-emitting circuit 11 and the light-receiving circuit 12 are disposed on the same side of the heart rate measurement device 100, the light received by the light-receiving circuit 12 is a light that is reflected back (i.e., a reflected light). If the light-emitting circuit 11 and the light-receiving circuit 12 are disposed on opposing sides of the heart rate measurement device 100, the light received by the light-receiving circuit 12 is a light that is refracted back (i.e., a refracted light).

In order to reduce an interference of an environment to the light emitted by the light-emitting circuit 11 in the heart rate detecting sensor 10 to thereby improve the accuracy of the PPG signal of the blood, the heart rate detecting sensor 10 is preferably disposed at a side of the heart rate measurement device 100 that is in contact with the skin of the user.

Herein, during a non-intense activity, because a relatively smaller portion of a green light is absorbed, thus the green light can be used to accurately detect the PPG signal of the blood during the non-intense activity. During an intense activity, because a relatively smaller portion of a red light is absorbed, thus the red light can be used to accurately detect the PPG signal of the blood during the intense activity.

Herein, two light-emitting sub-circuits are configured in the heart rate measurement device 100, which are respectively configured to emit a green light and a red light, thus the PPG signal of the blood can be accurately detected during different states of the movement (no-intense activity and/or intense activity), thereby ensuring that the heart rate is accurately measured.

In a second aspect, the present disclosure further provides a method for measuring a heart rate of a user by means of the heart rate measurement device according to any of the embodiments as described above. The detailed description of the method can be referenced to the following examples.

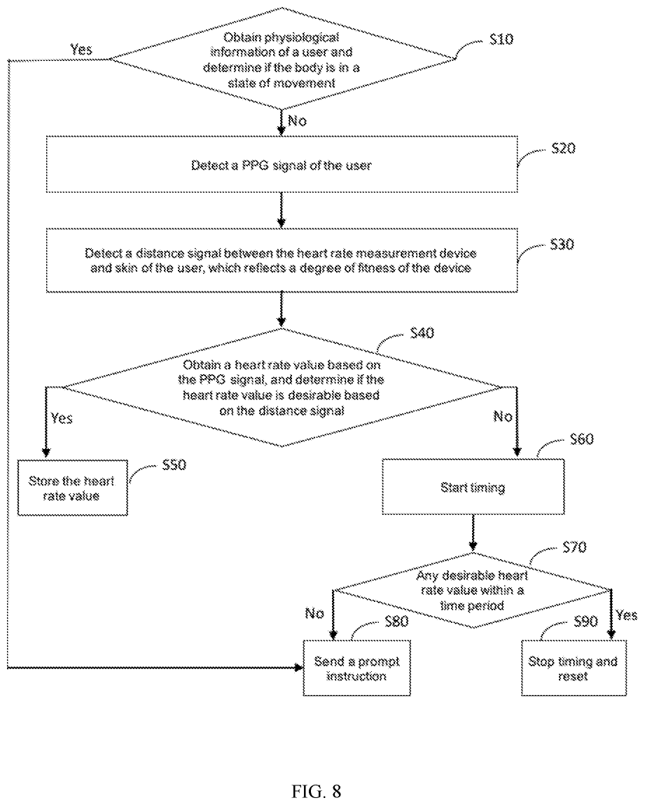

FIG. 8 shows a flow chart of a method for measuring a heart rate of a user by means of a heart rate measurement device according to some embodiments of the present disclosure. The method comprises:

S10: collecting physiological information of the user, determining whether a body of the user is in a state of movement based on the physiological information, and executing S80 if so, or executing S20 if otherwise;

Herein, the physiological information of the user, such as pulse rate, body temperature, and heart beats, is collected by a physiological information collecting portion 60.

S20: detecting a PPG signal of the user;

Herein, the PPG signal of the user can be detected by a sensor employing a PPG heart rate detection.

S30: detecting a distance between the heart rate measurement device 100 and skin of the user, wherein the distance reflects a degree of fitness between the heart rate measurement device 100 and the skin of the user.

Herein, the distance can be detected by a proximity sensor, a distance sensor, or a temperature sensor.

S40: obtaining a heart rate value based on the PPG signal, determining whether the heart rate value is desirable based on the distance, and executing S50 if so, or executing S60 if otherwise;

Herein, the processor 30 determines if the heart rate measurement device 100 is fit with the skin of the user by processing the distance, and further determines if the heart rate values is desirable.

Specifically, in the embodiment employing a temperature sensor for the detection of a distance between the heart rate measurement device 100 and the skin of the user, after receiving an instant temperature detected by the temperature sensor, the processor 30 obtains a temperature change by comparing the instant temperature with a reference temperature.

If the temperature change obtained by the processor 30 is greater than a preset threshold, the heart rate measuring device 100 is not fit with the skin of the user, the heart rate value that has been detected is not desirable, and then S60 is executed.

If the temperature change obtained by the processor 30 is smaller than a preset threshold, the heart rate measuring device 100 is closely fit with the skin of the user, the heart rate value that has been detected is desirable, and then S50 is executed.

Specifically, in the embodiment employing a proximity sensor for the detection of a distance between the heart rate measurement device 100 and the skin of the user, after receiving an instant optical pulse signal detected by the proximity sensor, the processor 30 compares the instant optical pulse signal with a reference optical pulse signal.

If a change of the optical pulse signal is greater than a preset threshold, the heart rate measuring device 100 is not fit with the skin of the user, the heart rate value that has been detected is not desirable, and then S60 is executed.

If the instant optical pulse signal is smaller than the preset threshold, the heart rate measuring device 100 is closely fit with the skin of the user, the heart rate value that has been detected is desirable, and then S50 is executed.

Specifically, in the embodiment employing a distance sensor for the detection of a distance between the heart rate measurement device 100 and the skin of the user, after receiving an instant time interval reflecting a distance between the heart rate measurement device 100 and the skin of the user that is detected by the distance sensor, the processor 30 compares the instant time interval with a preset threshold.

If the instant time interval is greater than a preset threshold, the heart rate measuring device 100 is not fit with the skin of the user, the heart rate value that has been detected is not desirable, and then S60 is executed.

If the instant time interval is smaller than the preset threshold, the heart rate measuring device 100 is closely fit with the skin of the user, the heart rate value that has been detected is desirable, and then S50 is executed.

S50: storing the heart rate value;

Herein the heart rate value can be stored in the memory 40 of the heart rate measurement device, or can be transmitted to an outside terminal through a wireless connection

S60: starting timing;

S70: determining whether there is at least one desirable heart rate value within a time period, and executing S90 if so, or executing S80 if otherwise;

S80: sending a prompt instruction;

S90: stopping timing and resetting.

Herein, the prompt instruction can be delivered via at least one of vibration, light, or sound, and can also be delivered by directly displaying the instant heart rate value. Through these prompt instructions, the user can be prompted to adjust the heart rate measurement device 100 such that it can be fit with the skin of the user.

Herein, there is no limitation to the execution of the heart rate measurement device 100 if none of the heart rate value that has been obtained is desirable. For example, the erroneous heart rate value can be deleted, or can be compensated through a compensation algorithm.

If the prompt instruction is delivered to the user by displaying the prompt instruction, the method can further comprise:

displaying a heart rate value if there is at least one desirable heart rate value.

In the embodiment of the method as described above, by detecting a distance between the heart rate measurement device 100 and skin of a user to thereby examine a degree of fitness between the heart rate measurement device 100 and the skin of the user, it can be determined whether the heart rate value obtained based on the PPG signal is desirable.

If it is determined that the heart rate measurement device 100 is not fit with the skin of the user, the heart rate value is determined as inaccurate and is thus deleted. If it is determined that the heart rate measurement device 100 is fit with the skin of the user, the heart rate value is determined as accurate and is thus stored in the memory 40. As such, the inaccurate heart rate values are deleted and all the heart rate values stored in the memory 40 are accurate, thereby improving the accuracy of measuring the heart rates of the user.

By sending a prompt instruction to a user if a heart rate value that is just obtained is not desirable, the user can be prompted to adjust the heart rate measurement device 100 such that it can be fit with the skin of the user, thereby ensuring that the heart rate values can be accurately obtained, realizing a real-time monitoring of heart rate of the user.

By collecting the physiological information of the user, the heart rate measurement device 100 can thus intelligently recognize a state of movement of the user. If it is determined that the body of the user is in a state of movement, the user is prompted to adjust the heart rate measurement device 100 such that it can be fit with the skin of the user, thereby improving the accuracy of measuring heart rates of the user.

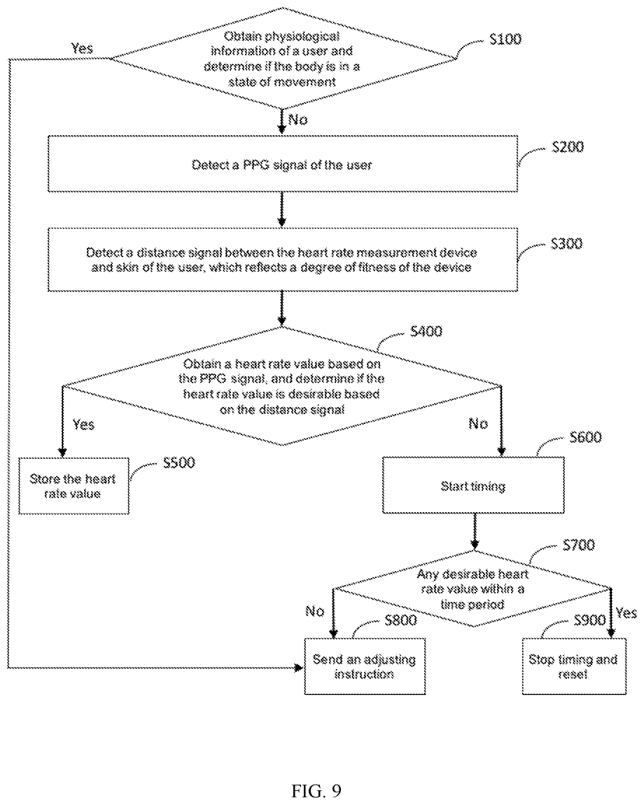

FIG. 9 shows a flow chart of a method for measuring a heart rate of a user by means of a heart rate measurement device according to some other embodiments of the present disclosure. The method comprises:

S100: collecting physiological information of the user, determining whether a body of the user is in a state of movement based on the physiological information, and executing S800 if so, or executing S200 if otherwise;

S200: detecting a PPG signal of the user;

S300: detecting a distance between the heart rate measurement device 100 and skin of the user, wherein the distance reflects a degree of fitness between the heart rate measurement device 100 and the skin of the user.

S400: obtaining a heart rate value based on the PPG signal, determining whether the heart rate value is desirable based on the distance, and executing S500 if so, or executing S600 if otherwise;

S500: storing the heart rate value;

S600: starting timing;

S700: determining whether there is at least one desirable heart rate value within a time period, and executing S900 if so, or executing S800 if otherwise;

S800: sending an adjusting instruction such that an adjusting portion adjusts the heart rate measurement device 100 to be fit with the skin of the user based on the adjusting instruction.

S900: stop timing and resetting.

Herein, the adjusting portion 70 of the heart rate measurement device 100 can receive an adjusting instruction to thereby automatically adjust the heart rate measurement device 100 to be fit with the skin of the user.

By sending an adjusting instruction if a heart rate value that is just obtained is not desirable, the adjusting portion 70 can be configured to automatically adjust the heart rate measurement device 100 to be fit with the skin of the user, thereby increasing the accuracy of measuring heart rates of the user, and further improving the comfortability of wearing.

It can be understood by those of ordinary skill in the art that all or part of the steps in the above mentioned embodiments of the method can be accomplished by means of hardware accompanied with program instructions, which may be stored in a computer-readable storage medium. Executing the program substantially comprises the steps of the method as described above.

Herein, the storage medium includes a variety of media, such as ROM, RAM, disk, or optical disk, which can store codes for the program.

In all of the embodiments of the device and the method as disclosed herein, the PPG-based heart rate determining approach is utilized for the determination of a heart rate of a user. It should be noted that in addition to the PPG-based approach, other approaches, including an ECG-based heart rate determining approach, a pulse oximetry-based heart rate determining approach, or an arterial pressure-based heart rate determining approach, can also be utilized for determining a heart rate of the user.

There are no limitations herein.

All references cited in the present disclosure are incorporated by reference in their entirety. Although specific embodiments have been described above in detail, the description is merely for purposes of illustration. It should be appreciated, therefore, that many aspects described above are not intended as required or essential elements unless explicitly stated otherwise.

Various modifications of, and equivalent acts corresponding to, the disclosed aspects of the exemplary embodiments, in addition to those described above, can be made by a person of ordinary skill in the art, having the benefit of the present disclosure, without departing from the spirit and scope of the disclosure defined in the following claims, the scope of which is to be accorded the broadest interpretation so as to encompass such modifications and equivalent structures.

1. A method for monitoring heart rates of a user wearing a heart rate measurement device configured to detect biometric signals of the user, each at a first time interval, the method comprising, each time when a biometric signal is to be detected:
detecting one biometric signal of the user and a distance signal reflecting and corresponding to a degree of fitness between the heart rate measurement device and skin of the user;
obtaining a heart rate value based on the one biometric signal, and determining whether the heart rate value is desirable based on the distance signal; and
keeping the heart rate value if the heart rate value is determined to be desirable, or discarding the heart rate value if otherwise.

2. (canceled)

3. (canceled)

4. The method of claim 1, wherein in the keeping the heart rate value if the heart rate value is determined to be desirable, or discarding the heart rate value if otherwise, the discarding the heart rate value comprises:
starting timing; and
sending an action instruction to the heart rate measurement device to adjust the degree of fitness between the heart rate measurement device and the skin of the user if no heart rate value is determined as desirable within a time period, or stopping timing and resetting if otherwise.

5. The method of claim 4, wherein:
the action instruction comprises a prompt instruction configured to prompt the user for manually adjusting the heart rate measurement device to be fit with the skin of the user; and
the sending an action instruction to the heart rate measurement device to adjust the degree of fitness between the heart rate measurement device and the skin of the user comprises:
sending the prompt instruction to the heart rate measurement device.

6. The method of claim 4, wherein:
the action instruction comprises an adjusting instruction configured to instruct the heart rate measurement device for automatically adjusting to be fit with the skin of the user; and
the sending an instruction to the heart rate measurement device to adjust the degree of fitness between the heart rate measurement device and the skin of the user comprises:
sending the adjusting instruction to the heart rate measurement device.

7. The method of claim 1, further comprising, prior to the detecting one biometric signal of the user and a distance signal reflecting and corresponding to a degree of fitness between the heart rate measurement device and skin of the user:
collecting physiological information of the user;
determining whether a body of the user is in a state of movement based on the physiological information; and
sending an action instruction to the heart rate measurement device to adjust the degree of fitness between the heart rate measurement device and the skin of the user if the body of the user is determined to be in the state of movement.

8. The method of claim 7, wherein:
the action instruction comprises a prompt instruction configured to prompt the user for manually adjusting the heart rate measurement device to be fit with the skin of the user; and
the sending an action instruction to the heart rate measurement device to manually or automatically adjust the degree of fitness between the heart rate measurement device and the skin of the user comprises:
sending the prompt instruction to the heart rate measurement device.

9. The method of claim 7, wherein:
the action instruction comprises an adjusting instruction configured to instruct the heart rate measurement device for automatically adjusting to be fit with the skin of the user; and
the sending an instruction to the heart rate measurement device to manually or automatically adjust the degree of fitness between the heart rate measurement device and the skin of the user comprises:
sending the adjusting instruction to the heart rate measurement device.

10. (canceled)

11. The method of claim 1, wherein the distance signal comprises a temperature signal, wherein:
the detecting one biometric signal of the user and a distance signal reflecting and corresponding to a degree of fitness between the heart rate measurement device and skin of the user comprises:
detecting an instant temperature of the user by a temperature sensor; and
the obtaining a heart rate value based on the one biometric signal and determining whether the heart rate value is desirable based on the distance signal comprises:
obtaining a change of temperature by comparing the instant temperature with a reference temperature; and
determining that the heart rate value is not desirable if the change of temperature is greater than a preset threshold, or determining that the heart rate value is desirable if otherwise.

12. The method of claim 1, wherein the distance signal comprises an optical pulse signal, wherein:
the detecting one biometric signal of the user and a distance signal reflecting and corresponding to a degree of fitness between the heart rate measurement device and skin of the user comprises:
detecting an instant optical pulse signal reflected from the skin of the user by a proximity sensor; and
the obtaining a heart rate value based on the one biometric signal and determining whether the heart rate value is desirable based on the distance signal comprises:
obtaining a change of optical pulse signal by comparing the instant optical pulse signal with a reference optical pulse signal; and
determining that the heart rate value is not desirable if the change of optical pulse signal is greater than a preset threshold, or determining that the heart rate value is desirable if otherwise.

13. The method of claim 1, wherein the distance signal comprises a second time interval signal, wherein:
the detecting one biometric signal of the user and a distance signal reflecting and corresponding to a degree of fitness between the heart rate measurement device and skin of the user comprises:
detecting an instant second time interval signal of an infrared light reflected from the skin of the user by a distance sensor; and
the obtaining a heart rate value based on the one biometric signal and determining whether the heart rate value is desirable based on the distance signal comprises:
comparing the instant second time interval signal with a preset threshold; and
determining that the heart rate value is not desirable if the instant second time interval signal is greater than the preset threshold, or determining that the heart rate value is desirable if otherwise.

14. A heart rate measurement device, comprising a heart rate detecting sensor, a fitness detecting sensor, and a processor, wherein:
the heart rate detecting sensor is coupled to the processor, and is configured to detect, and to transmit to the processor, one biometric signal of a user at a first time interval;
the fitness detecting sensor is coupled to the processor, and is configured to detect, and to transmit to the processor, a distance signal corresponding to the one biometric signal, wherein the distance signal reflects and corresponds to a degree of fitness between the heart rate measurement device and the skin of the user; and
the processor is configured to obtain a heart rate value based on the one biometric signal, and to determine whether the heart rate value is desirable based on the distance signal.

15. (canceled)

16. The heart rate measurement device of claim 14, further comprising an action portion coupled to the processor, wherein:
the processor is further configured to start timing if determining that the heart rate value is not desirable, and to send an action instruction to the action portion if no heart rate value is determined as desirable within a time period, or to stop timing and reset if otherwise; and
the action portion is configured, upon receiving the action instruction from the processor, to act on the heart rate measurement device to adjust the degree of fitness between the heart rate measurement device and the skin of the user.

17. The heart rate measurement device of claim 16, wherein the action portion comprises a prompting portion, and the action instruction comprises a prompt instruction, wherein:
the prompting portion is configured, upon receiving the prompt instruction from the processor, to prompt the user for manually adjusting the heart rate measurement device to be fit with the skin of the user.

18. The heart rate measurement device of claim 16, wherein the action portion comprises an adjusting portion, and the action instruction comprises an adjusting instruction, wherein:
the adjusting portion is configured, upon receiving the adjusting instruction from the processor, to automatically adjust the heart rate measurement device to be fit with the skin of the user.

19. The heart rate measurement device of claim 14, further comprising a physiological information collecting portion and an action portion, each coupled to the processor, wherein:
the physiological information collecting portion is configured to collect physiological information of the user, and to send the physiological information to the processor; and
the processor is further configured to determine whether a body of the user is in a state of movement based on the physiological information, and if so, to start timing and to send an action instruction to the action portion, or if otherwise, to stop timing and reset; and
the action portion is configured, upon receiving the action instruction from the processor, to act on the heart rate measurement device to adjust the degree of fitness between the heart rate measurement device and the skin of the user.

20. The heart rate measurement device of claim 19, wherein the action portion comprises a prompting portion, and the action instruction comprises a prompt instruction, wherein:
the prompting portion is configured, upon receiving the prompt instruction from the processor, to prompt the user for manually adjusting the heart rate measurement device to be fit with the skin of the user.

21. The heart rate measurement device of claim 19, wherein the action portion comprises an adjusting portion, and the action instruction comprises an adjusting instruction, wherein:
the adjusting portion is configured, upon receiving the adjusting instruction from the processor, to automatically adjust the heart rate measurement device to be fit with the skin of the user.

22.-25. (canceled)

26. The heart rate measurement device of claim 14, wherein the fitness detecting sensor comprises a temperature sensor, wherein:
the temperature sensor is configured to detect an instant temperature of the user; and
the processor is further configured to obtain a change of temperature by comparing the instant temperature with a reference temperature, and to determine that the heart rate value is not desirable if the change of temperature is greater than a preset threshold, or that the heart rate value is desirable if otherwise.

27. The heart rate measurement device of claim 14, wherein the fitness detecting sensor comprises a proximity sensor, wherein:
the proximity sensor is configured to detect an instant optical pulse signal reflected from the skin of the user; and
the processor is further configured to obtain a change of optical pulse signal by comparing the instant optical pulse signal with a reference optical pulse signal, and to determine that the heart rate value is not desirable if the change of optical pulse signal is greater than a preset threshold, or that the heart rate value is desirable if otherwise.

28. The heart rate measurement device of claim 14, wherein the fitness detecting sensor comprises a distance sensor, wherein:
the distance sensor is configured to detect an instant second time interval signal of an infrared light reflected from the skin of the user; and
the processor is further configured to compare the instant second time interval signal with a preset threshold, and to determine that the heart rate value is not desirable if the instant second time interval signal is greater than the preset threshold, or that the heart rate value is desirable if otherwise.

29. (canceled)
